(19) United States
(12) Patent Application Publication
(10) Pub. No.: US 2006/0253062 A1
(43) Pub. Date: Nov. 9, 2006

(54) Low resistance irrigation system and apparatus

(75) Inventors: Grace C. Liao, Irvine, CA (US); George Brody, San Clemente, CA (US); Mel M. Oliveira, Huntington Beach, CA (US)

Correspondence Address: ALCON, IP LEGAL, TB4-8, 6201 SOUTH FREEWAY, FORT WORTH, TX, 76134, US

(73) Assignee: ALCON, INC.

(21) Appl. No.: 11/114,514

(22) Filed: Apr. 26, 2005

(51) Int. Cl. A61M 1/00

(52) U.S. Cl. 604/27

(57) ABSTRACT

A surgical irrigation system having reduced irrigation flow resistance. Reduction in irrigation fluid flow resistance is achieved by increasing the diameter of the irrigation fluid tubings. The ends of the tubings are tapered to reduce the stiffness of the tubings and to allow the tubings to be connected to current surgical devices.

### BACKGROUND OF THE INVENTION

This invention relates generally to the field of cataract surgery and more particularly to a control system for a phacoemulsification handpiece.

The human eye in its simplest terms functions to provide vision by transmitting light through a clear outer portion called the cornea, and focusing the image by way of the lens onto the retina. The quality of the focused image depends on many factors including the size and shape of the eye, and the transparency of the cornea and lens.

When age or disease causes the lens to become less transparent, vision deteriorates because of the diminished light which can be transmitted to the retina. This deficiency in the lens of the eye is medically known as a cataract. An accepted treatment for this condition is surgical removal of the lens and replacement of the lens function by an artificial intraocular lens (IOL).

In the United States, the majority of cataractous lenses are removed by a surgical technique called phacoemulsification. During this procedure, a thin phacoemulsification cutting tip is inserted into the diseased lens and vibrated ultrasonically. The vibrating cutting tip liquifies or emulsifies the lens so that the lens may be aspirated out of the eye. The diseased lens, once removed, is replaced by an artificial lens.

A typical ultrasonic surgical device suitable for ophthalmic procedures consists of an ultrasonically driven handpiece, an attached cutting tip, and irrigating sleeve and an electronic control console. The handpiece assembly is attached to the control console by an electric cable and flexible fluid tubings. Through the electric cable, the console varies the power level transmitted by the handpiece to the attached cutting tip and the flexible fluid tubings supply irrigation fluid to and draw aspiration fluid from the eye through the handpiece assembly.

The operative part of the handpiece is a centrally located, hollow resonating bar or horn directly attached to a set of piezoelectric crystals. The crystals supply the required ultrasonic vibration needed to drive both the horn and the attached cutting tip during phacoemulsification and are controlled by the console. The crystal/horn assembly is suspended within the hollow body or shell of the handpiece by flexible mountings. The handpiece body terminates in a reduced diameter portion or nosecone at the body's distal end. The nosecone is externally threaded to accept the irrigation sleeve. Likewise, the horn bore is internally threaded at its distal end to receive the external threads of the cutting tip. The irrigation sleeve also has an internally threaded bore that is screwed onto the external threads of the nosecone. The cutting tip is adjusted so that the tip projects only a predetermined amount past the open end of the irrigating sleeve. Ultrasonic handpieces and cutting tips are more fully described in U.S. Pat. Nos. 3,589,363; 4,223,676; 4,246,902; 4,493,694; 4,515,583; 4,589,415; 4,609,368; 4,869,715; 4,922,902; 4,989,583; 5,154,694 and 5,359,996, the entire contents of which are incorporated herein by reference.

In use, the ends of the cutting tip and irrigating sleeve are inserted into a small incision of predetermined width in the cornea, sclera, or other location. The cutting tip is ultrasonically vibrated along its longitudinal axis within the irrigating sleeve by the crystal-driven ultrasonic horn, thereby emulsifying the selected tissue in situ. The hollow bore of the cutting tip communicates with the bore in the horn that in turn communicates with the aspiration line from the handpiece to the console. A reduced pressure or vacuum source in the console draws or aspirates the emulsified tissue from the eye through the open end of the cutting tip, the cutting tip and horn bores and the aspiration line and into a collection device. The aspiration of emulsified tissue is aided by a saline flushing solution or irrigant that is injected into the surgical site through the small annular gap between the inside surface of the irrigating sleeve and the cutting tip.

The preferred surgical technique is to make the incision into the anterior chamber of the eye as small as possible in order to reduce the risk of induced astigmatism. These small incisions result in very tight wounds that squeeze the irrigating sleeve. Such a tight wound construction decreases the stability of the eye, particularly when high aspiration vacuums (above 500 mm Hg) and/or high flows (in excess of 40 cc/min.) are used, because changes in the irrigation flow caused by either changes in the aspiration flow rate or by rapid changes in aspiration vacuum cannot be damped by the inflow of irrigation fluid, which is restricted. Theoretically, increasing the amount of irrigating fluid entering the eye will help to stabilize the intraocular pressure (“IOP”); however, in a clinical setting, the amount of irrigation fluid entering the eye is limited to the amount of fluid aspirated from the eye due to the tight wound construction with minimal leakage from the wound. Also, increasing the flow of irrigating fluid through the eye increases the turbulence in the eye, possibly leading to endothelial cell loss, postoperative inflammation and edema.

Therefore, a need continues to exist for a system that helps to maintain a stable IOP even at high aspiration vacuum levels.

### BRIEF SUMMARY OF THE INVENTION

The present invention improves upon the prior art by providing a surgical irrigation system having reduced irrigation flow resistance. Reduction in irrigation fluid flow resistance is achieved by increasing the diameter of the irrigation fluid tubings. The ends of the tubings are tapered to reduce the stiffness of the tubings and to allow the tubings to be connected to current surgical devices.

Accordingly, one objective of the present invention is to provide a surgical irrigation system having reduced irrigation flow resistance.

Another objective of the present invention is to provide a surgical irrigation system having more stable intraocular pressures.

Another objective of the present invention is to provide a surgical irrigation system that allows for higher aspiration vacuum.

Another objective of the present invention is to provide a surgical irrigation system that allows for higher aspiration flow.

These and other advantages and objectives of the present invention will become apparent from the detailed description and claims that follow.

### BRIEF DESCRIPTION OF THE DRAWINGS

FIG. 1 is a perspective view of a handpiece and control console that may be used with the present invention.

FIG. 2 is a schematical representation of the handpiece and control console illustrated in FIG. 1.

FIG. 3 is a transverse cross-sectional view of the cutting tip of the present invention taken at line 3-3 in FIG. 2.

### DETAILED DESCRIPTION OF THE INVENTION

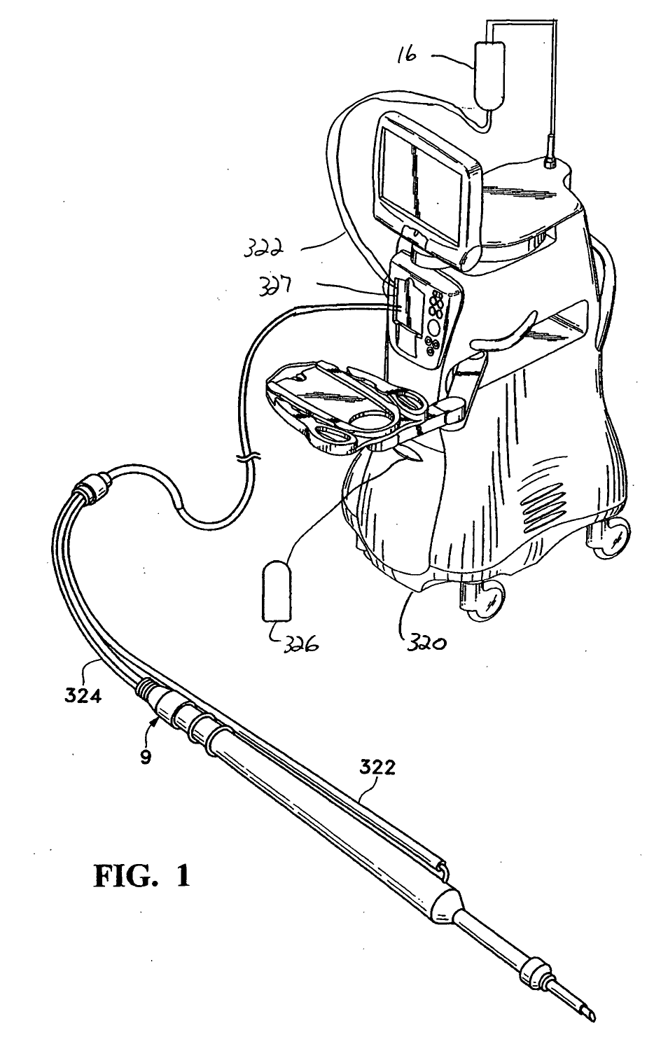

As best seen in FIG. 1, surgical console 320 suitable for use with the present invention may be any commercially available surgical control console such as the INFINITI® surgical systems available from Alcon Laboratories, Inc., Fort Worth, Tex. Console 320 is connected to handpiece 9 through irrigation line 322 and aspiration line 324, and the flow through lines 322 and 324 is controlled by the user, for example, via footswitch 326.

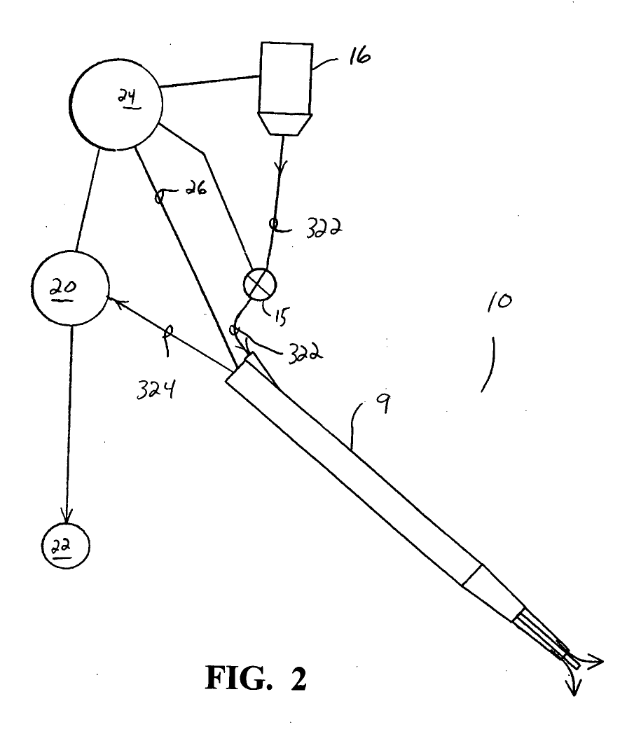

As seen in FIG. 2, schematically, system 10 embodied in console 320 that may be used in the present invention generally included handpiece 9, which is supplied with irrigating fluid through tubings 322 from source 16. Tubings 322 may contain check valve 15 or some other suitable device for controlling the flow of irrigating fluid in tubings 322. The infusion fluid from source 16 is pressurized either by gravity or by pressurizing source 16. Aspiration line 324 fluidly connects handpiece 12 to pump 20, which aspiration fluid for a surgical site and empties the aspirated fluid into container 22. Handpiece 9 is also electronically connected to control module 24 by cable 26. Control module 24 is contained within console 320 and operates to control aspiration pump 20, infusion source 16, valve 15 and the power supplied to handpiece 12.

Change in the intraocular pressure is directly proportional to the irrigation fluid flow resistance in the irrigation system. Therefore, by reducing the irrigation fluid flow resistance in the irrigation system, a more stable IOP can be maintained, even at high aspiration vacuums, without increased irrigation fluid flow. This reduction in the irrigation fluid flow resistance in the irrigation system is best accomplished by increasing the internal and external of tubings 322. For example, using irrigation tubings 322 having an internal diameter of between approximately 0.150 inches and 0.250 inches, with approximately 0.190 inches being preferred and having an external diameter of between approximately 0.190 inches and 0.300 inches, with approximately 0.281 inches being preferred, allows for vastly increased irrigation fluid free flow rates (up to approximately 148 cc/min) indicating greatly reduced resistance to flow in tubings 322. One drawback of using such large diameter tubings is that current fittings used on cassettes, check valves, handpieces, etc., are sized to be used with smaller I.D. and O.D. tubing. Increasing the diameters of tubings 322 requires the fitting on all devices in the fluid pathway to be redesigned and/or resized. In addition, larger diameter tubing is stiffer than smaller diameter tubing. Stiffening tubing 322 where it connects to handpiece 9 makes handpiece 9 more difficult to manipulate, resulting in decreased feel and mobility, which is undesirable.

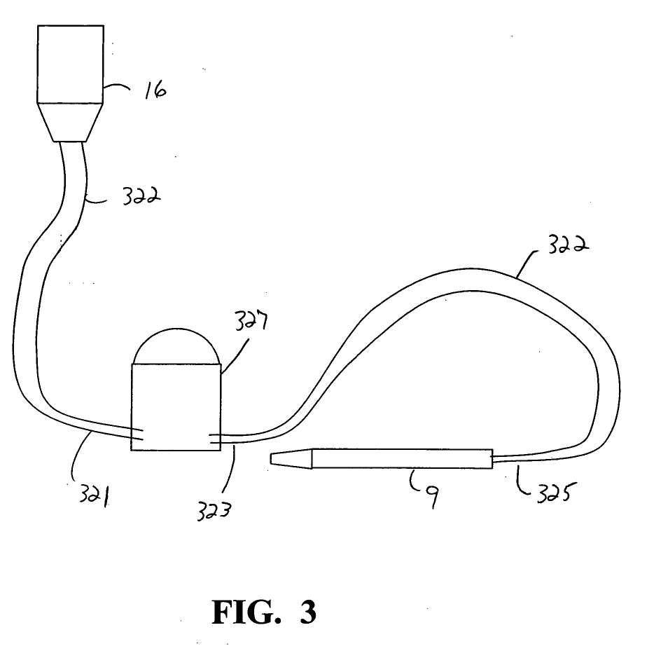

As best seen in FIG. 3, the inventors have discovered a way to increase the diameter of tubing without corresponding decrease in handpiece mobility by reducing the diameter of tubings 322 at portions 321, 323 and 325 where tubings 322 connect to cassette 327 and handpiece 9. For example, portions 321, 323 and 325 may have an internal diameter of between approximately 0.060 inches and 0.180 inches, with approximately 0.160 inches being preferred and having an external diameter of between approximately 0.090 inches and 0.200 inches, with approximately 0.190 inches being preferred. Such reduction in diameter allows tubings 322 to be more easily connected to conventional cassette 327 and handpiece 9 without modification and decreases the stiffness of portion 325 near handpiece 9. Tubings 322 are of increased interior and exterior diameters along a substantial portion of the length of tubings 322, with reduced diameter portions 321, 323 and 325 making up on a relatively short portion of the length of tubings 322, for example, between approximately 12.0 inches and 24.0 inches.

This description is given for purposes of illustration and explanation. It will be apparent to those skilled in the relevant art that changes and modifications may be made to the invention described above without departing from its scope or spirit.

We claim:

1. A surgical system, comprising:
a) a surgical console;
b) a source of irrigation fluid associated with the surgical console;
c) a surgical handpiece; and
d) a fluid tubing having a distal end and a proximal end, the fluid tubing connected to the surgical handpiece at the distal end and connected to the source of irrigation fluid at the proximal end, the fluid tubing having a first internal diameter at the proximal end, the first internal diameter extending along a substantial portion of a length of the fluid tubing, the fluid tubing further having a second internal diameter less than the first internal diameter along a portion of the fluid tubing at the distal end of the fluid tubing, the portion of the fluid tubing having the second internal diameter being shorter than the substantial portion of the length of the fluid tubing having the first internal diameter.

2. A fluid tubing having a distal end and a proximal end, the fluid tubing capable of connecting to a surgical handpiece at the distal end and to a source of irrigation fluid at the proximal end, the fluid tubing having a first internal diameter at the proximal end, the first internal diameter extending along a substantial portion of a length of the fluid tubing, the fluid tubing further having a second internal diameter less than the first internal diameter along a portion of the fluid tubing at the distal end of the fluid tubing, the portion of the fluid tubing having the second internal diameter being shorter than the substantial portion of the length of the fluid tubing having the first internal diameter.

3. The surgical system of claim 1 where the first internal diameter is between approximately 0.150 inches and 0.250 inches.

4. The surgical system of claim 1 where the second internal diameter is between approximately 0.190 inches and 0.300 inches.
